(19) United States
(12) Patent Application Publication
(10) Pub. No.: US 2021/0121085 A1
(43) Pub. Date: Apr. 29, 2021

(54) Sphygmomanometer Arm Band and Control Method Thereof

(71) Applicant: AViTA Corporation, New Taipei City (TW)

(72) Inventors: Hsing Ou Yang, New Taipei City (TW); JUI-HSINAG LIN, New Taipei City (TW)

(21) Appl. No.: 17/074,545

(22) Filed: Oct. 19, 2020

(30) Foreign Application Priority Data
Oct. 18, 2019 (TW) 108213812

(51) Int. Cl. A61B 5/022

(52) U.S. Cl. CPC A61B 5/02233

(57) ABSTRACT

A sphygmomanometer arm band for an arm-type sphygmomanometer includes a cuff and a clamp. The clamp includes an arc-grip part, a first arc-shaped force portion, and a second arc-shaped force portion, wherein the arc-grip part is configured to elastically clamp on the cuff, wherein the first arc-shaped force portion and the second arc-shaped force portion are respectively disposed on the outer side of the arc-grip part for applying a force to control a gap between the two opposite ends of the arc-grip part. Thereby, the blood pressure measurement operation can be performed more conveniently and the measurement result can be more accurate.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a technical field of medical instrument, and more particularly, to a sphygmomanometer arm band and a control method thereof.

### Description of Related Arts

Blood pressure is an important vital sign for human health and indicates the functional state of the human heart, blood vessels and blood circulation system, and is an important evidence for clinical identification of diseases, examination of medical effects, and prognostic prediction.

With the advancement of science, people's life quality is improving day by day, and the accelerating pace of life makes modern people pay more and more attention to their own health issues. Measuring blood pressure helps people understand their own health conditions and prevent the danger of diseases. Therefore, the use of sphygmomanometers has become more and more common.

The arm-type sphygmomanometers, which are convenience in measurement, have become more and more popular. The arm band of the conventional arm-type clamped sphygmomanometer mainly includes a two-piece structure similar to a claw clip, and the two-piece structure includes the hub and spring disposed between two clip parts. When in use, the hub and spring, which are obviously protruded, may cause discomfort on the subject's upper arm in contact. Furthermore, during the process of inflation and deflation of the cuff, the conventional two-piece structure may cause the blood pressure measurement result to be less accurate.

Therefore, the present invention provides a sphygmomanometer arm band and a control method thereof to solve the above-mentioned problems.

### SUMMARY OF THE PRESENT INVENTION

An objective of the present invention is to provide a sphygmomanometer arm band for an arm-type sphygmomanometer, and the sphygmomanometer arm band includes a cuff and a clamp. The clamp includes an arc-grip part having two opposite ends and configured to elastically clamp on the cuff, and a first arc-shaped force portion and a second arc-shaped force portion disposed on outer sides of the two opposite ends of the arc-grip part and configured to control a gap between the two opposite ends of the arc-grip part.

According to an embodiment, the first arc-shaped force portion and the second arc-shaped force portion have a first handle part and a second handle part extended toward an outer side of the arc-grip part respectively, and an interval is formed between the first handle part and the second handle part, wherein the interval is distant from the gap of the two opposite ends. A curvature of the first arc-shaped force portion is equal to a curvature of the second arc-shaped force portion, and a curvature radius of the arc-grip part is less than or equal to a curvature radius of one of the first arc-shaped force portion and the second arc-shaped force portion.

According to an embodiment, the arc-grip part is made by elastic material; the arc-grip part, the first arc-shaped force portion and the second arc-shaped force portion can be made by the same material; an elastic coefficient of the arc-grip part is less than or equal to an elastic coefficient of one of the first arc-shaped force portion and the second arc-shaped force portion.

According to an embodiment, the cuff can include a cloth cover having a first glove opening and a second glove opening, wherein the first glove opening and the second glove opening of the cloth cover are configured to sleeve the two opposite ends of the arc-grip part respectively, and make the arc-grip part clamp on the cuff, wherein the first handle part of the first arc-shaped force portion and the second handle part of the second arc-shaped force portion are exposed out of the first glove opening and the second glove opening respectively.

Another objective of the present invention is to provide a control method of the sphygmomanometer arm band, wherein the sphygmomanometer arm band includes a cuff and a clamp including an arc-grip part, and a first arc-shaped force portion and a second arc-shaped force portion disposed on outer sides of the two opposite ends of the arc-grip part, wherein the control method includes steps of: providing a first handle part and a second handle part formed on the first arc-shaped force portion and the second arc-shaped force portion and extended toward an outer side of the arc-grip part respectively, wherein an interval is formed between the first handle part and the second handle part, and the interval is distant from the gap between the two opposite ends; and when an external force is applied on the first handle part and the second handle part, using the first arc-shaped force portion and the second arc-shaped force portion to make the gap between the two opposite ends of the arc-grip part pass a subject's arm therethrough, and then elastically clamping the arc-grip part on the arm, so as to attach the cuff on the subject's arm.

According to the present invention, the configuration of the arc-grip part, the first arc-shaped force portion and the second arc-shaped force portion enable the blood pressure measurement operation to be performed more conveniently and make the measurement result more accurate.

### BRIEF DESCRIPTION OF THE DRAWINGS

The structure, operating principle and effects of the present invention will be described in detail by way of various embodiments which are illustrated in the accompanying drawings.

FIG. 1 is a schematic structural view of a sphygmomanometer arm band, according to a first embodiment of the present invention.

FIG. 2 is a sectional view of a sphygmomanometer arm band of FIG. 1 not being applied by an external force, according to the above first embodiment of the present invention.

FIG. 3 is a schematic structural view of a clamp of a sphygmomanometer arm band of FIG. 1.

FIG. 4 is a side view of the clamp of FIG. 3 not being applied by an external force.

FIG. 5 is a schematic structural view of the clamp of FIG. 3 being applied by an external force.

FIG. 6 is a schematic structural view of a sphygmomanometer arm band, according to a second embodiment of the present invention.

FIGS. 7A and 7B are side view and perspective view of the arc-shaped force portion of FIG. 6.

### DETAILED DESCRIPTION OF THE PREFERRED EMBODIMENT

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

Spatially relative terms, such as “central”, “lateral”, “above”, “upper”, “beneath”, “below”, “lower”, “left”, “right”, “vertical”, “level”, “top”, “bottom”, “inside”, “outside” and the like, may be used herein for ease of description to describe one element or feature's relationship to another element(s) or feature(s) as illustrated in the figures, and may not necessarily mean that an element or a device must be positioned on a specific orientation. It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be fixedly, detachably or integrally, mechanically, electrically, or directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the terms “comprise”, “include”, “have” and variations such as “comprises”, “comprising”, “includes”, “including”, “has”, and/or “having” used in the present specification will imply the existence of stated shapes, numbers, steps, operations, elements, and/or groups thereof, but does not the exclusion of any other shapes, numbers, steps, operations, elements, and/or groups thereof.

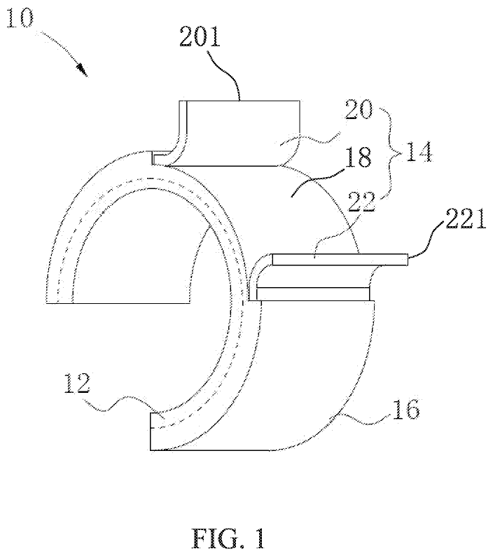

Please refer to FIG. 1, which is a schematic structural view of a sphygmomanometer arm band 10, according to a first embodiment of the present invention. The first embodiment is described for explanation of at least one advantage of the present invention, as shown in FIG. 1, the sphygmomanometer arm band 10 for an arm-type sphygmomanometer includes a cuff 12 and a clamp 14. The cuff 12 includes a cloth cover 16, and the clamp 14 is disposed on the cuff 12 and includes an arc-grip part 18, a first arc-shaped force portion 20, and a second arc-shaped force portion 22. The arc-grip part 18 has two opposite ends 24, and a gap is formed between the two opposite ends 24. Preferably, the gap can have a curvature of 90 degrees, but the present invention is not limited thereto.

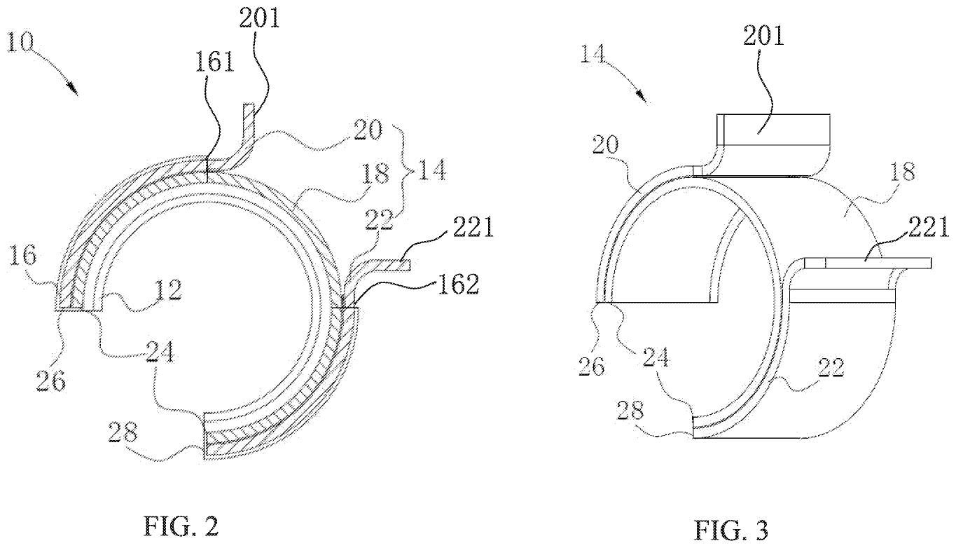

Please refer to FIGS. 1 and 2. FIG. 2 is a sectional view of FIG. 1. As shown in FIG. 2, the clamp 14 includes the arc-grip part 18, the first arc-shaped force portion 20 and the second arc-shaped force portion 22. In an embodiment of the present invention, the first arc-shaped force portion 20 and the second arc-shaped force portion 22 are fixedly disposed on outer surfaces of the two opposite ends 24 of the arc-grip part 18 respectively. The arc-grip part 18 is configured to elastically clamp on the cuff 12, to make the cuff 12 tightly attach on a subject's arm. The cloth cover 16 of the cuff 12 is configured to cover the arc-grip part 18, the first arc-shaped force portion 20 and the second arc-shaped force portion 22, and the cloth cover 16 has a first glove opening 161 and a second glove opening 162 configured to respectively sleeve the two opposite ends 24 of the arc-grip part 18, to make the arc-grip part 18 clamp on the cuff 12. A first handle part 201 of the first arc-shaped force portion 20 and a second handle part 221 of the second arc-shaped force portion 22 are exposed out of the first glove opening 161 and the second glove opening 162 respectively.

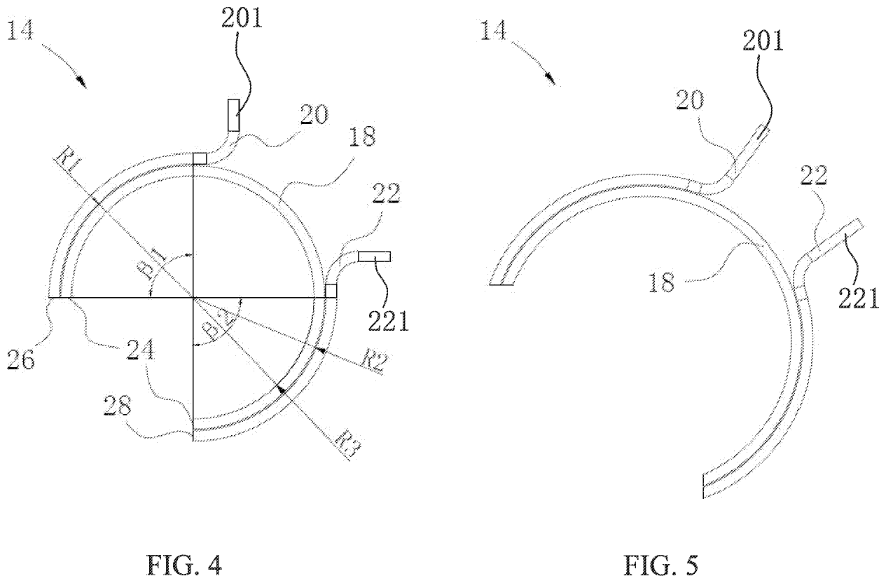

Since the end 26 of the first arc-shaped force portion 20 and the end 28 of the second arc-shaped force portion 22 are fixed on the two opposite ends 24 of the arc-grip part 18 respectively, the first arc-shaped force portion 20 and the second arc-shaped force portion 22 can be coupled to the two opposite ends 24 of the arc-grip part 18. The first arc-shaped force portion 20 and the second arc-shaped force portion 22 have the first handle part 201 and the second handle part 221 extended toward the outer side of the arc-grip part 18 respectively. An interval is formed between the first handle part 201 and the second handle part 221, and distant from the gap between the two opposite ends 24 of the arc-grip part 18. As shown in FIG. 5, a user can apply force on the first handle part 201 of the first arc-shaped force portion 20 and the second handle part 221 of the second arc-shaped force portion 22, to control the gap between the two opposite ends 24 of the arc-grip part 18, so as to allow the subject's arm to pass through the gap. When the force is removed, as shown in FIG. 4, the arc-grip part 18 can be elastically clamped on the cuff 12, to make the cuff 12 tightly attach on the subject's arm.

However, the manner of connecting the first arc-shaped force portion 20 and the second arc-shaped force portion 22 and the two opposite ends 24 of the arc-grip part 18 is not limited to the above-mentioned example. For example, the first arc-shaped force portion 20 and the second arc-shaped force portion 22 can also be directly connected to the two opposite ends 24 of the arc-grip part 18 by a locking manner, an adhering manner, or a buckling manner. In another embodiment, the first arc-shaped force portion 20, the second arc-shaped force portion 22 and the arc-grip part 18 can be formed integrally.

Please refer to FIGS. 1 and 3. FIG. 3 is a schematic structural view of the clamp 14 of the sphygmomanometer arm band 10, according to the present invention. As shown in FIG. 3, the clamp 14 includes the first arc-shaped force portion 20, the second arc-shaped force portion 22, and the arc-grip part 18. The end 26 of the first arc-shaped force portion 20 and the end 28 of the second arc-shaped force portion 22 are aligned to the two opposite ends 24 of the arc-grip part 18, respectively, so as to form the gap. The interval is formed between the first handle part 201 of the first arc-shaped force portion 20 and the second handle part 221 of the second arc-shaped force portion 22 and distant from the gap between the two opposite ends 24. When a force is applied on the first arc-shaped force portion 20 and the second arc-shaped force portion 22, the first handle part 201 of the first arc-shaped force portion 20 and the second handle part 221 of the second arc-shaped force portion 22 are moved close to each other, so as to open the gap between the two opposite ends 24 of the arc-grip part 18, to facilitate the subject's arm to put through the gap. After the external force is removed, the curved inner surface of the arc-grip part 18 can make the cuff 12 attach on the subject's arm more closely, so that the measurement result can be more accurate.

Please refer to FIG. 4, which is a side view of the clamp of FIG. 3 not being applied by external force. In this embodiment, a curvature (31 of the first arc-shaped force portion 20 is equal to a curvature (32 of the second arc-shaped force portion 22, a curvature radius R1 of the first arc-shaped force portion 20 is equal to a curvature radius R2 of the second arc-shaped force portion 22, and a curvature radius R1 of the first arc-shaped force portion 20 is larger than a curvature radius R3 of the arc-grip part 18. However, the present invention is not limited the above-mentioned example, and in other implementations, each of the curvature radius R1 of the first arc-shaped force portion 20 and the curvature radius R2 of the second arc-shaped force portion 22 can be equal to the curvature radius R3 of the arc-grip part 18; or the curvature radius of one of the first arc-shaped force portion 20 and the second arc-shaped force portion 22 is larger than the curvature radius R3 of the arc-grip part 18; or the curvature radius of one of the first arc-shaped force portion 20 and the second arc-shaped force portion 22 is equal to the curvature radius R3 of the arc-grip part 18. In other words, any condition in which the first handle part 201 of the first arc-shaped force portion 20 and the second handle part 221 of the second arc-shaped force portion 22 can be used to effectively apply force on the two ends 24 of the arc-grip part 18, is within the scope of the present invention.

It is to further explain that the arc-grip part 18, the first arc-shaped force portion 20 and the second arc-shaped force portion 22 can be made by elastic material, to enable the clamp 14 to effectively clamp on the subject's arm, so as to make the cuff 12 attach on the subject's arm. Furthermore, the first arc-shaped force portion 20, the second arc-shaped force portion 22 and the arc-grip part 18 can be made by the same material.

Please refer to FIGS. 4 and 5. FIG. 5 is a schematic structural view of the clamp 14 of FIG. 3 being applied by external force. As shown in FIG. 5, when an external force is applied on the first handle part 201 of the first arc-shaped force portion 20 and the second handle part 221 of the second arc-shaped force portion 22, the first arc-shaped force portion 20, the second arc-shaped force portion 22 and the arc-grip part 18 are deformed. In this embodiment, an elastic coefficient of the arc-grip part 18 is less than an elastic coefficient of each of the first arc-shaped force portion 20 and the second arc-shaped force portion 22, so that the external force applied on the first arc-shaped force portion 20 and the second arc-shaped force portion 22 can be effectively transferred to the arc-grip part 18, so that the gap between the two ends 24 of the arc-grip part 18 can be opened easier to facilitate a subject to put arm into the clamp 14 when the clamp 14 is in use. However, the present invention is not limited to the above-mentioned example, and in another embodiment, the elastic coefficient of each of the first arc-shaped force portion 20 and the second arc-shaped force portion 22 can be equal to the elastic coefficient of the arc-grip part 18, or the elastic coefficient of one of the first arc-shaped force portion 20 and the second arc-shaped force portion 22 can be larger than the elastic coefficient R3 of the arc-grip part 18, or the elastic coefficient of one of the first arc-shaped force portion 20 and the second arc-shaped force portion 22 can be equal to the elastic coefficient R3 of the arc-grip part 18. In other words, any condition in which the first handle part 201 of the first arc-shaped force portion 20 and the second handle part 221 of the second arc-shaped force portion 22 can open the gap between the two end 24 of the arc-grip part 18 to facilitate the subject to put arm into the clamp 14, can be adapted to the present invention.

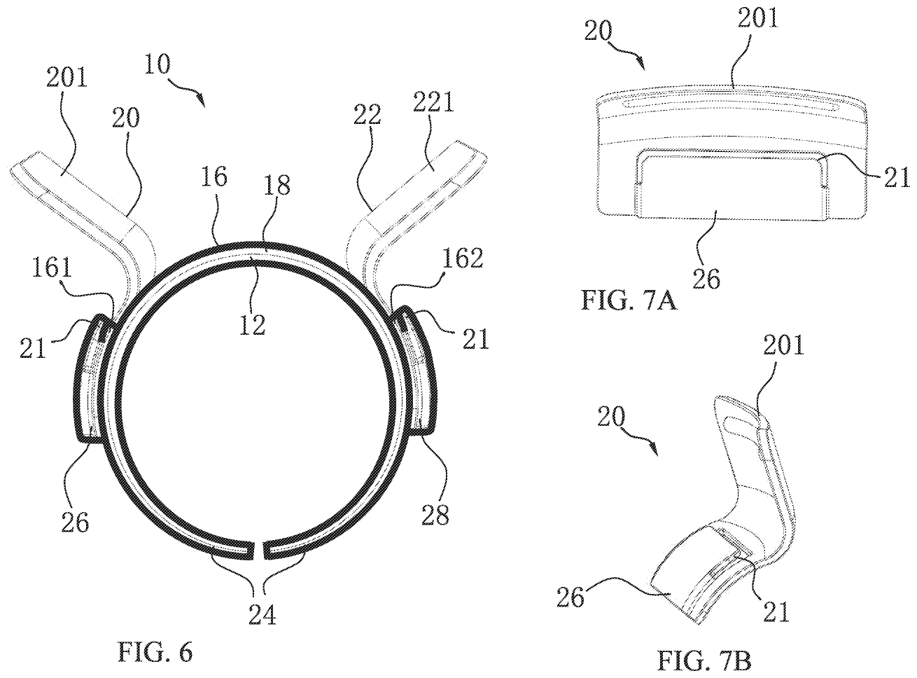

Please refer to FIG. 6, which is a schematic structural view of the sphygmomanometer arm band 10, according to a second embodiment of the present invention. According to this embodiment of the present invention, the sphygmomanometer arm band 10 includes a cuff 12 and a clamp, and the clamp includes an arc-grip part 18, a first arc-shaped force portion 20 and a second arc-shaped force portion 22. The arc-grip part 18 has two opposite ends 24, and a gap is formed between the two opposite ends 24. The cuff 12 includes a cloth cover 16 configured to tightly enclose the arc-grip part 18 of the clamp, so that the cuff 12 can be located on the inner side of the arc-grip part 18. Compared with the above first embodiment in which the first arc-shaped force portion 20 and the second arc-shaped force portion 22 are fixedly disposed on the outer surface of the arc-grip part 18, the first arc-shaped force portion 20 and the second arc-shaped force portion 22 according to the above second embodiment of the present invention are detachably mounted on the outer side of the cloth cover 16. The cloth cover 16 enclosing the arc-grip part 18 has a first glove opening 161 and a second glove opening 162 which are located on the outer surface of the arc-grip part 18 and configured to sleeve the end 26 of the first arc-shaped force portion 20 and the end 28 of the second arc-shaped force portion 22 respectively. With the configuration of tightly enclosing the arc-grip part 18 by the cloth cover 16, an external force can be applied to the first handle part 201 of the first arc-shaped force portion 20 and the second handle part 221 of the second arc-shaped force portion 22, so as to control the gap between the two opposite ends 24 of the arc-grip part 18 to allow the subject's arm to smoothly pass therethrough. When the external force is removed, the arc-grip part 18 is elastically clamped on the cuff 12, to make the cuff 12 tightly attach with the subject's arm.

According to the above second embodiment of the present invention, the first arc-shaped force portion 20 and the second arc-shaped force portion 22, which are detachable, can have the same structures and curve contours. The first arc-shaped force portion 20 is taken as example for illustration in following paragraph. FIGS. 7A and 7B show a side view and a perspective view of the first arc-shaped force portion 20. The first arc-shaped force portion 20 has a first handle part 201 and an end 26 which are formed integrally, and the end 26 of the first arc-shaped force portion has the peripheral curvature of the arc-grip part 18, and the first handle part 201 is radially extended from the end 26 of the first arc-shaped force portion to the arc-grip part 18. The first arc-shaped force portion has a reverse hook structure 21 formed on the end 26, and when the end 26 of the first arc-shaped force portion is sleeved into the first glove opening 161 of the cloth cover 16, the reverse hook structure 21 can hook the edge of the first glove opening 161, to maintain the end 26 of the first arc-shaped force portion 20 to locate inside the first glove opening 161. Similarly, the second arc-shaped force portion 22 having the same structure as the first arc-shaped force portion 20 can be sleeved into the second glove opening 162 of the cloth cover 16 by the same way.

In accordance with an embodiment of the present invention, a control method for a sphygmomanometer arm band includes the following steps. The cuff 12 is combined with the clamp 14, and the clamp 14 includes the arc-grip part 18, and the first arc-shaped force portion 20 and the second arc-shaped force portion 22 disposed on outer side of the two opposite ends 24 of the arc-grip part 18 respectively. The arc-grip part 18 is configured to elastically clamp on the cuff 12 and has a gap formed between the two opposite ends 24. The first arc-shaped force portion 20 and the second arc-shaped force portion 22 can be fixedly disposed on the outer surface of the arc-grip part 18 (such as the first embodiment of the present invention), or the cloth cover 16 can be used to tightly enclose the arc-grip part 18 and the first arc-shaped force portion 20 and the second arc-shaped force portion 22, which are detachable, are sleeved into the first glove opening 161 and the second glove opening 162 of the cloth cover 16. The first handle part 201 and the second handle part 221 are formed on the first arc-shaped force portion 20 and the second arc-shaped force portion 22 and extended toward outer side of the arc-grip part 18 respectively. The interval is formed between the first handle part 201 and the second handle part 221 and is distant from the gap of the two opposite ends 24. When the first handle part 201 and the second handle part 221 are controlled by an external force, the first arc-shaped force portion 20 and the second arc-shaped force portion 22 can control the gap between the two opposite ends 24 of the arc-grip part 18 to facilitate a subject's arm to pass therethrough, so as to make the arc-grip part 18 elastically clamp on the subject's arm and attach the cuff 12 on the arm.

It is to further explain that the sphygmomanometer arm band 10 can include a rubber hose (not shown in figures) and a sphygmomanometer (not shown in figures). The rubber hose is connected to the sphygmomanometer and the cuff 12, and the sphygmomanometer can inflate and deflate the cuff 12 through the rubber hose. The inflation and deflation operations can be performed manually, or the sphygmomanometer can automatically inflate or deflate the cuff 12. In another embodiment, the sphygmomanometer can include a blood pressure calculation component (not shown in figures), and when the inflation and deflation for the cuff 12 is completed, the blood pressure calculation component can calculate the subject's blood pressure value, and show the information of blood pressure value on a display (not shown in figures).

In order to measure the blood pressure, the first arc-shaped force portion 20 and the second arc-shaped force portion 22 can be forced to open the arc-grip part 18 to directly clamp on the subject's arm, and the sphygmomanometer can be used to inflate and deflate the cuff 12 and calculate the subject's blood pressure value.

According to above-mentioned contents of the sphygmomanometer arm band 10 of the present invention, configuration of the arc-grip part 18, the first arc-shaped force portion 20 and the second arc-shaped force portion 22 can prevent from compressing the subject's arm to make the subject uncomfortable, and enable the blood pressure measurement operation to be performed more conveniently and make the measurement result more accurate.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A sphygmomanometer arm band, comprising:
a cuff; and
a clamp comprising:
an arc-grip part having two opposite ends and configured to elastically clamp on the cuff; and
a first arc-shaped force portion and a second arc-shaped force portion disposed on outer sides of the two opposite ends of the arc-grip part and configured to control a gap between the two opposite ends of the arc-grip part.

2. The sphygmomanometer arm band, according to claim 1, wherein the first arc-shaped force portion and the second arc-shaped force portion have a first handle part and a second handle part extended toward an outer side of the arc-grip part respectively, wherein an interval is formed between the first handle part and the second handle part, wherein the interval is distant from the gap of the two opposite ends.

3. The sphygmomanometer arm band, according to claim 2, wherein when an external force is applied on the first handle part and the second handle part, the first arc-shaped force portion and the second arc-shaped force portion control the gap between the two opposite ends of the arc-grip part.

4. The sphygmomanometer arm band, according to claim 1, wherein a curvature radius of the arc-grip part is less than or equal to a curvature radius of one of the first arc-shaped force portion and the second arc-shaped force portion.

5. The sphygmomanometer arm band, according to claim 1, wherein the arc-grip part is made of elastic material.

6. The sphygmomanometer arm band, according to claim 5, wherein an elastic coefficient of the arc-grip part is less than or equal to an elastic coefficient of one of the first arc-shaped force portion and the second arc-shaped force portion.

7. The sphygmomanometer arm band, according to claim 1, wherein an elastic coefficient of the arc-grip part is less than or equal to an elastic coefficient of one of the first arc-shaped force portion and the second arc-shaped force portion.

8. The sphygmomanometer arm band, according to claim 1, wherein the cuff comprises a cloth cover having a first glove opening and a second glove opening, wherein the first glove opening and the second glove opening of the cloth cover are configured to sleeve the two ends of the first arc-shaped force portion and the second arc-shaped force portion respectively, and make the arc-grip part clamp on the cuff, wherein the first handle part of the first arc-shaped force portion and the second handle part of the second arc-shaped force portion are exposed out of the first glove opening and the second glove opening respectively.

9. The sphygmomanometer arm band, according to claim 1, wherein the gap between the two opposite ends of the arc-grip part has a curvature of 90 degrees.

10. A control method of a sphygmomanometer arm band of claim 1, comprising the following steps:
providing a first handle part and a second handle part formed on the first arc-shaped force portion and the second arc-shaped force portion and extended toward an outer side of the arc-grip part respectively, wherein an interval is formed between the first handle part and the second handle part, and the interval is distant from the gap between the two opposite ends; and
when an external force is applied on the first handle part and the second handle part, using the first arc-shaped force portion and the second arc-shaped force portion to make the gap between the two opposite ends of the arc-grip part pass a subject's arm therethrough, and then elastically clamping the arc-grip part on the arm, so as to attach the cuff on the subject's arm.
